(19) United States
(12) Patent Application Publication
(10) Pub. No.: US 2023/0419724 A1
(43) Pub. Date: Dec. 28, 2023

(54) AUTOMATED CAMERA-BASED NON-INVASIVE MONITORING FOR SPONTANEOUS BREATHING AND AWAKENING TRIALS IN INTENSIVE CARE

(71) Applicant: KONINKLIJKE PHILIPS N.V., Eindhoven (NL)

(72) Inventors: Daniel Jason Schulman, Cambridge, MA (US); Haibo Wang, Cambridge, MA (US); Cornelis van Zon, Cambridge, MA (US)

(21) Appl. No.: 18/209,202

(22) Filed: Jun. 13, 2023

Related U.S. Application Data
(60) Provisional application No. 63/354,256, filed on Jun. 22, 2022.

(51) Int. Cl. G06V 40/16; G06V 20/52; G06V 40/20; G06V 10/94; A61B 5/00

(52) U.S. Cl. CPC G06V 40/176; G06V 20/52; G06V 40/20; G06V 40/172; G06V 10/945; A61B 5/0077; G16H 10/60

(57) ABSTRACT

An embodiment provides objective measures or indications to facilitate evaluation of readiness and successful progress or completion of spontaneous breathing and awakening trials (SATs/SABs) in intensive care settings. One example includes obtaining a patient state indicating that the patient is associated with a SAT or SBT protocol. Imagery of the patient for the protocol is obtained and evaluated to classify the imagery as indicative of an observation item associated with the protocol. An indication based on the evaluating is provided, for example to a clinical decision support system.

### CROSS-REFERENCE TO RELATED APPLICATIONS

This patent application claims the priority benefit under 35 U.S.C. § 119(e) of U.S. Provisional Application No. 63/354,256, filed on Jun. 22, 2022, the contents of which are herein incorporated by reference.

### BACKGROUND OF THE INVENTION

### 1. Field of the Invention

The disclosed subject matter generally pertains to clinical decision support systems and related technology. Some of the disclosed subject matter pertains to technology that provides objective measures and indications to facilitate evaluation of readiness or successful progress or completion of spontaneous breathing and awakening trials in intensive care settings.

### 2. Description of the Related Art

Mechanical ventilation may be used within an intensive care unit (ICU) when a patient is unable to breathe independently. A widely used mechanical ventilation technique is invasive ventilation, providing access to the patient's lower airways through tracheostomy or an endotracheal tube.

Removal of ventilation support from a patient is important. However, proper timing of removal trials is conventionally elusive. Incorrect timing leads to trial failure and renewed reliance on ventilation. Ventilation removal failures may lead to poor patient outcomes.

Nonetheless, attempting to remove ventilation support as soon as possible remains desirable. For most patients on mechanical ventilation in intensive care, oversedation and prolonged ventilation are associated with poor outcomes. Clinical guidelines for pain, agitation/sedation, delirium, immobility, and sleep disruption (PADIS) management in the ICU, and the associated “ABCDEF” implementation strategy of the Society of Critical Care Medicine, recommend for most patients daily spontaneous awakening trials (SATs) and spontaneous breathing trials (SBTs) as a key part of a strategy for “ICU Liberation” and for improved patient outcomes.

In SATs the use of sedatives is temporarily interrupted, and the patient is observed to assess whether they regain consciousness. During an SAT, the patient is observed to determine whether signs of potential adverse events are present. If these signs occur, the SAT is a failure and sedation may be resumed. In SBTs, ventilator support is reduced to minimal, and the patient is observed to assess whether they resume normal breathing. Again, during a SBT the patient is observed to determine whether signs of potential adverse events are present. If so, the SBT is a failure and full ventilation support is resumed. In current recommendations, SATs and SBTs are used together in a “wake up and breathe” protocol.

### SUMMARY OF THE INVENTION

Conventionally, adherence to protocols for SATs and SBTs can be difficult, which limits their effectiveness. In one study while use of SATs and SBTs separately had high adherence (as measured by % of patient-days they were performed), coordinated adherence (i.e., a SAT followed by an SBT) was much poorer, and adherence decreased with higher workload burden on clinical staff.

Accordingly, an embodiment provides objective measures or indications of observation items, such as pain, agitation, and anxiety associated with a wake up and breathe protocol or parts thereof, i.e., SAT safety screening, SAT, SBT safety screening, and SBT. An embodiment therefore facilitates evaluation of readiness and successful progress or completion of spontaneous breathing and awakening trials in intensive care settings using objective data and repeatable processes.

In summary, an embodiment includes a method, comprising: obtaining, using a set of one or more processors, a patient state indicating that a patient is associated with one or more of a spontaneous awakening trial (SAT) and a spontaneous breathing trial (SBT); obtaining, using a set of one or more sensors, imagery of the patient associated with one or more of the SAT and the SBT; evaluating, using the set of one or more processors, the imagery of the patient to classify the imagery as indicative of an observation item associated with one or more of the SAT and SBT; and providing, using the set of one or more processors, an indication based on the evaluating.

In an embodiment, the imagery comprises facial imagery and the evaluating comprises classifying the facial imagery as indicative of an observation item including one or more of pain, agitation, and anxiety. The imagery may be obtained in a continuous manner during the protocol.

In an embodiment, the set of one or more sensors provide sensor data indicative of one or more of patient movement and body posture; and the evaluating comprises using the sensor data and the imagery to classify the patient as ready for one or more of a SAT and a SBT or as successfully completing or failing an SAT or SBT.

An embodiment displays the indication or data derived therefrom in an interactive display. The interactive display comprises a display component having one or more interactive elements associated with points in the wake up and breathe protocol or part thereof. The one or more interactive elements comprise a link to the imagery or data derived therefrom. In an embodiment, the display comprises the indication incorporated into an electronic health record. In an embodiment, the indication is provided to an end point including one or more of an in-room patient display, a clinician mobile device, a centralized monitoring station for an ICU, a remotely located telemedicine facility, and a scheduling system.

An embodiment provides a device that performs the methods described herein. In an embodiment, the device, includes: a set of one or more processors; and a set of one or more memory devices operatively coupled to the set of one or more processors and storing code executable by the set of one or more processors to: obtain a patient state indicating that a patient is associated with one or more of a spontaneous awakening trial (SAT) and a spontaneous breathing trial (SBT); obtain imagery of the patient associated with one or more of the SAT and the SBT; evaluate the imagery of the patient to classify the imagery as indicative of an observation item associated with one or more of the SAT and SBT; and provide an indication based on evaluating the imagery.

An embodiment provides a computer program product, comprising: a non-transitory storage medium comprising computer executable code, the computer executable code comprising: code that obtains (201), using a set of one or more processors, a patient state indicating that a patient is associated with one or more of a spontaneous awakening trial (SAT) and a spontaneous breathing trial (SBT); code that obtains (203) imagery of the patient associated with one or more of the SAT and the SBT; code that evaluates (204) the imagery of the patient to classify the imagery as indicative of an observation item associated with one or more of the SAT and SBT; and code that provides (205) an indication based on evaluating the imagery.

The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an example wake up and breathe protocol.

FIG. 2 is an example method of providing an objective indication of an observation item associated with a wake up and breathe protocol or part thereof.

FIG. 3 is an example system.

FIG. 4 is an example interactive display.

FIG. 5 is a diagram of example system components.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “operatively coupled” means that two or more elements are coupled so as to operate together or are in communication, unidirectional or bidirectional, with one another. As used herein, the term “number” shall mean one or an integer greater than one (i.e., a plurality). As used herein a “set” shall mean one or more.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

Conventional approaches to implementation of guidelines for ventilator liberation rely heavily on periodic monitoring and assessment by clinical staff of various observation items used as part of a wake up and breathe protocol or part thereof. Typically, clinical staff such as nurses or respiratory therapists monitor and assess the patient to make judgments about these observation items. While many of the assessed observation items are objectively available from chart data and vital signs monitoring, some observation items are assessed subjectively by clinical staff. The observation items that rely on clinical staff monitoring and subjective judgment include determining absence of agitation for SAT safety screening, presence of anxiety, agitation, or pain for SAT failure assessment, absence of agitation for SBT safety screening, and mental status change for SBT failure assessment.

As there are several observation items that rely on clinical staff monitoring, difficulties with conventional protocols include increased workload burden for clinical staff, difficulties in scheduling appropriate clinical staff to make the observations, difficulty in performing scheduled observations when urgent response to adverse events (with the same or other patients) is needed, greater difficulty in performing SATs and SBTs, and the subjectivity of the assessments of the observation items, which may contribute to false negatives.

It will be more fully appreciated by reference to this detailed description, its examples, and the associated drawings that in an embodiment, an automated, camera-based assessment of a patient supplements or replaces one or more observation items used in a wake up and breathe protocol or part thereof, i.e., for conducting SATs and SBTs. For example, an embodiment provides a continuous, computer-vision based assessment of patient images to detect pain and agitation and supplies a resultant indication for use in a wake up and breathe protocol, e.g., to a clinical decision support system or scheduling system endpoint.

An embodiment therefore promotes semi-automation of a wake up and breathe protocol, including automated assessment of pain, anxiety, or agitation. The automated assessment can be reviewed and confirmed by clinical staff, allows for improved screening of patient readiness, and lessens the burden placed on clinical staff by SATs and SBTs. An embodiment's assessment also contributes to accurately determining signs of SAT and SBT failure, decreasing the effort for clinical staff, particularly when ICU burden is high. An embodiment reduces or eliminates variability in evaluating observation items by supplying consistency in assessment of observation items. In an embodiment, continuous assessment, for example over several hours or a 24-hour period, or other window of time associated with a protocol or part thereof, allows for capturing all relevant data related to accurately assessing observation items in a protocol. Further, trends in observation item assessment may be provided to clinical staff, aiding in final resolution of any ambiguities that may be observed.

The description now turns to the figures. The illustrated example embodiments will be best understood by reference to the figures. The following description is intended only by way of example, and simply illustrates certain example embodiments.

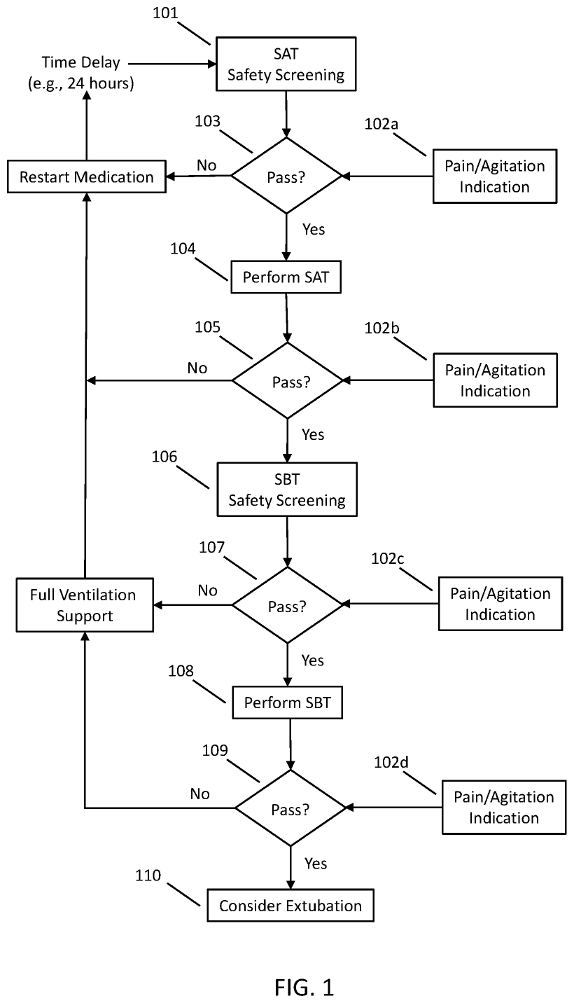

FIG. 1 illustrates an example wake up and breathe protocol that includes SAT and SBT related parts or subroutines. In the example of FIG. 1, SAT safety screening is performed at 101 to evaluate a patient's readiness to conduct the SAT at 104. The safety screening may include, for example, review of medical chart information, vital information from health sensors, as well as review of observation items related to the patient's appearance to determine observation items indicative of readiness for SAT. By way of specific example, SAT safety screening 101 may include determination that the patient is not suffering from active seizures, is not being withdrawn from alcohol, is not agitated, is not on certain medication (e.g., paralytics), is not experiencing myocardial ischemia, and has normal intercranial pressure. A negative conclusion as to one or more of these items may indicate that the patient is not ready for the SAT at 104 and therefore a conclusion may be made that the patient has not passed the SAT safety screening, as indicated at 103. In such a case, medication may be restarted, and the patient awaits a new attempt window, e.g., the next day.

It may be appreciated that among observation items utilized in SAT safety screening at 101, determining that the patient is not agitated is subjective and relies on clinical staff's judgment for evaluation. In such a case, a variety of factors may contribute to inconsistency and inaccuracy, e.g., clinical staff experience, workload burden currently placed on clinical staff, availability of time and information to make an informed judgment regarding agitation, such as enough time to make meaningful observations, etc. In one conventional approach, a standardized assessment tool such as a critical care pain observation tool (CPOT) may be utilized, where clinical staff observes the patent and provides input to a scoring tool using various observation item parameters such as features of facial expression (e.g., relaxed/neutral, tense, grimacing), features of body movement (e.g., absence of movement, protection, restlessness), and muscle tension (e.g., relaxed, tense/rigid, or very tense/rigid).

An embodiment provides an objective indication of an observation item, e.g., detected agitation, or lack thereof, at 102a using an automated evaluation process. By way of example, an embodiment provides the indication at 102a based on evaluating image data of the patient. An embodiment may evaluate image data of the patient's face, body, related structures such as an endotracheal tube, or a combination of the foregoing. In one example, patient imagery may be obtained from a video stream showing, for example, full body imagery of the patient. Note that use of full body video allows assessment of both facial expression and body posture and movement. By way of specific example, an embodiment may utilize a computer vision process to evaluate facial imagery of a patient according to the Prkachin and Solomon Pain Index (PSPI) using selected facial action coding system (FACS) features, such as brow lowerer, cheek raiser, lid tightener and eyes closed, which remove FACS features related to the mouth (as it is occluded or partially occluded by the intubation tube). A resultant score from such image evaluation may be compared directly to a threshold used to indicate the presence or absence of a target condition or characteristic such as the observation item of a SAT safety screening protocol, e.g., agitation. In some examples, the threshold may be relative, e.g., to the patient or relevant comparison population or standard, and adapted over time, such as during a 24-hour evaluation window proceeding the SAT safety screening at 101. Further, a current score may be compared to a trend to indicate if the score is improving (showing less agitation), getting worse (showing more agitation), remaining stable, etc., each of which may be used in a determination as to the presence of agitation or like characteristic of interest.

In an embodiment, the use of facial image scoring may be supplemented or replaced by using other data, such as body posture data, medical chart data, patient wearable sensor data, equipment sensor data or imagery of associated structures, e.g., information indicating movement or change in position of an endotracheal tube. In one example embodiment, a variety of data is fused to make a determination regarding patient agitation and supply the indication of 102a. By way of example, two or more of body posture data, medical chart data, patient wearable sensor data, equipment sensor data and imagery of associated structures are supplied to a trained model to provide a classification, such as agitated/not agitated, which may or may not be associated with a score. A model used to evaluate patient imagery and/or other sensor data may be trained by providing manually, semi-automatically, or automatically labeled data to a classification program, such as a neural network, to provide the model with reference data for training rounds. Once a sufficiently trained model is obtained, it may be provided to a variety of devices for use in a process such as outlined in FIG. 1, as further described herein.

If an embodiment provides an indication at 102a that the patient is not experiencing agitation, which may include no indication or signal being provided, or is experiencing agitation within an acceptable threshold, the wake up and breathe protocol may progress to performance of the SAT at 104. The indication of 102a of FIG. 1 may be provided in a variety of ways to a variety of endpoints. For example, when a suitable program is integrated into in-room hospital equipment or electronic health record (EHR) system, the indication may be displayed on a bedside monitor. Also, an embodiment may choose an endpoint, such as a mobile device of clinical staff or a centralized device, e.g., a ward-level device showing statuses of a variety of patients within the ICU. Further, the indication may be provided as is, e.g., a raw score or classification result, or may be combined with other observation item(s) to form a clinical decision or suggestion, such as a readiness score or judgment indication that combines or weights a set of readiness criteria.

In a similar manner as evaluating readiness for the SAT at 303, an embodiment may provide an indication 102b to assist in evaluating an SAT that is in progress, as indicated at 105. The indication 102b may take a variety of forms and in at least one embodiment may be similar to the indication 102a. However, the observation items useful in evaluating different portions or time windows of the wake up and breathe protocol may differ. For example, an embodiment may continue to utilize the same or similar model to evaluate facial imagery of the patient during the SAT 104. Alternatively or additionally, an embodiment may utilize a different model, e.g., tuned via training data to utilize different image features relevant to identifying observation item(s) associated with a given time window of the protocol, e.g., an ongoing SAT 104, such as evaluating pain, agitation, or anxiety, or weighting one more features more heavily, e.g., signs of acute pain, co-occurrence of agitation, pain or anxiety and another feature such as arrythmia, respiratory distress, oxygen saturation below 88%, respiratory rate outside of a range, e.g., 8-35, 8-25, 10-18, 12-20, or 12-25 breaths per minute, etc., observed during an SAT 104. It is noted that any thresholds are provided as examples and different protocols may use different thresholds. Again, if an embodiment provides an indication that the patient is experiencing pain, agitation, anxiety or other relevant characteristic, the wake up and breathe protocol may be halted as an SAT failure, as indicated in FIG. 1.

Following a successful SAT safety screening 101 and SAT 104, an embodiment may similarly supplement conventional SBT safety screening 106. As may be appreciated, an embodiment may utilize the same or similar model as utilized to provide the indication 102b or may utilize a model tuned to features relevant to the protocol time window associated with observation items needed for evaluation of readiness for an SBT. An embodiment may provide an indication 102c to signal the presence or absence of a feature or characteristic of interest, e.g., agitation, for evaluation of SBT readiness at 107. Again, this indication 102c may be formed or used alone or may be formed or used in combination with other data, such as oxygen saturation level, fraction of inspired oxygen (FiO₂) level (e.g., below 50%), positive end-expiratory pressure (PEEP) level (e.g., below or equal to 7.5 cm H₂O), myocardial ischemia, vasopressor use, and quality of inspiratory efforts. For example, when indication 102c is used alone, indication of agitation alone may halt the wake and breathe protocol due to lack of confidence in SBT safety screening criteria being met. Alternatively, or in addition, the indication may be taken into consideration in combination with other data of interest to provide a readiness score for SBT. If indication 102c alone or in combination with other data is indicative of agitation or lack of readiness, the wake up and breathe protocol may be halted, as indicated at 107. Otherwise, a SBT may begin as shown at 108.

Similar to SAT safety screening 101, SBT safety screening 106, and SAT 104, at or following the beginning of the SBT 108, an embodiment may provide an indication 102d that assists in determining if the SBT is proceeding successfully or should be halted, as indicated at 109. As may be appreciated, an embodiment may utilize the same or similar model as utilized to provide the indication 102c or may utilize a model tuned to features relevant to identifying the state of observation item(s) for an ongoing SBT. By way of example, a model trained to detect change in pain or mental status may be particularly helpful in evaluating whether a SBT 108 is progressing well or should be halted. In making the determination at 109, data indicative of a change in mental status may be useful to aid, supplement or replace the judgment of clinical staff. A model for determining a change in mental status may be formed by supplying the model implementation, such as a neural network, with labeled training data that labels imagery indicative of a change in mental status. As with other protocol parts or time periods, an embodiment may train a model using labelled end result imagery (e.g., facial imagery from successful and unsuccessful SBTs) to permit a broader classification result (i.e., prediction of successful SBT) as opposed to attempting to provide a specific indication of change in mental status as indication 102d. If the indication 102d signals that SBT is progressing well, determination 109 may indicate that the SBT is successful and extubation may be considered as indicated at 110. Otherwise, the protocol may loop as indicated.

As illustrated in the example wake up and breathe protocol of FIG. 1, an embodiment supplements conventional approaches to further refine the accuracy and consistency of the protocols in terms of adherence to ideal standards. Current approaches to implementation of guidelines for ICU liberation rely heavily on periodic monitoring and assessment of certain subjective observation items by clinical staff. While many of the assessment items are available from objective chart data or vital signs readings, some are assessed with subjective observations from clinical staff including absence of agitation for SAT safety screening 101, presence of anxiety, agitation, or pain for SAT failure assessment 105, absence of agitation for SBT safety screening 106, and mental status change for SBT failure assessment 109. An embodiment addresses problems with conventional approaches by supplying objective model(s) for evaluating patient data as part of the workflow.

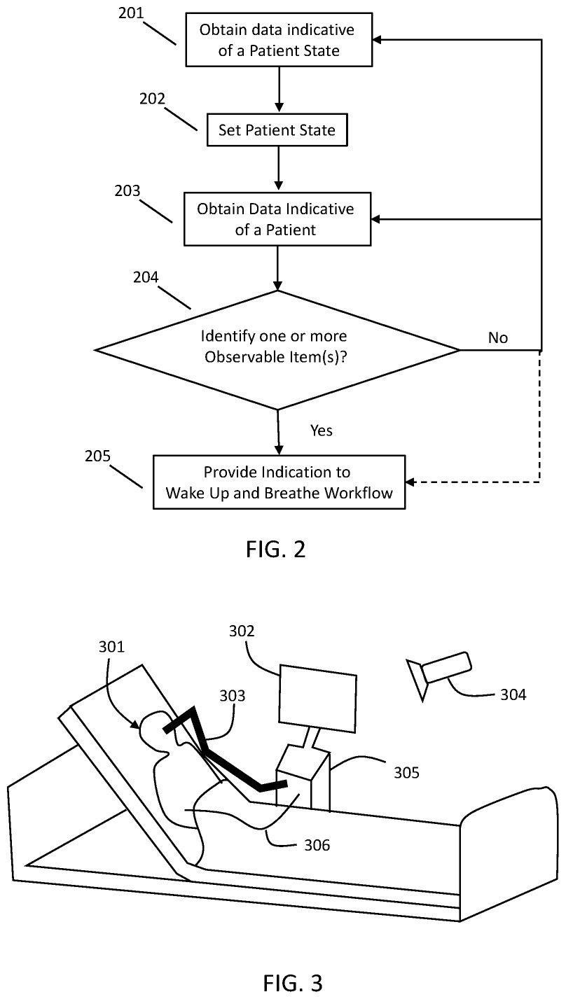

Turning to FIG. 2, an embodiment supplements existing assessments with continuous, automated, camera-based assessment of observation items of interest, such as pain and agitation, via computer vision. In an embodiment, semi-automated safety screening for SATs and SBTs can be performed, with an automatic assessment of the absence of agitation confirmed by clinical staff. Less effort should be required by clinical staff, decreasing workload burden and workflow difficulty. For example, as shown in FIG. 2, at 201 an embodiment obtains data indicative of a patient, workflow, or protocol state, e.g., patient is associated with an evaluation for SAT safety screening, an active SAT, an evaluation for SBT, or an active SBT. In one example, a patient state may indicate that the patient is not currently eligible for SATs/SBTs (for example, due to an underlying condition which has not resolved), planned to have a SAT/SBT and ready for being monitored for readiness, in the process of a SAT (i.e., sedative has been decreased or stopped), or in the process of an SBT. This permits an embodiment to set the patient, workflow, or protocol state at 202, e.g., evaluation of the patient for SAT safety screening.

As described herein, identifying the relevant patient state permits an embodiment to focus evaluation on feature(s) or characteristic(s) of the patient useful in the evaluation or observation item(s) for the protocol part in question. Further, the patient state permits an embodiment to provide feedback data to an appropriate endpoint, such as a scheduling or patient orchestration system, which may utilize automated assessment data to adjust patient workflow stages, clinician scheduling, clinician alerts, etc.

An embodiment may perform an evaluation at 204 using a variety of statistical, machine learning, or rule-based methods. By way of specific example, following obtaining of the patient state at 202, an embodiment may load or otherwise access or make available a model trained to identify features for providing output regarding a relevant observation item, e.g., agitation detected from patient imagery using FACS features as described herein. Again, the model may rely on patient facial imagery alone or take into consideration data from a variety of sources, such as medical chart data available from an electronic medical record, real time vitals sensors, etc. As described herein, a model used to detect an observation item feature, such as agitation, may accept as input additional data, such as body posture, body movement, medical chart data, vital signs data from wearable sensors, or other sensor data from associated equipment, and make use of the same in classifying the data (or set of data) as indicative of the feature of interest, e.g., agitation for SAT safety screening.

If an embodiment determines at 204 that a feature or characteristic of interest is detected, e.g., model classifies patient imagery as indicative of agitation, the indication may be provided to a wake up and breathe workflow, such as provisioning of an alert, alarm, or notification of readiness score to an in-room monitor, mobile clinician device, or central coordinating station or ward-level device. In this regard, it should be noted that an embodiment may provide the indication to a variety of endpoints, including scheduling orchestration software that prioritizes and orders patients for SATs and SBTs based on readiness scores and other criteria such as clinician availability. In an embodiment, the scheduling of a patient may be promoted or delayed based on the indication provided at 205. Otherwise, e.g., if imagery of the patient does not indicate agitation within a SAT screening timeframe, no indication may be provided. Alternatively, a positive indication (absence of agitation) may be supplied, promoting the progression of the workflow and scheduling towards SAT.

In an embodiment, continuous automated assessment of signs of SAT/SBT safety screening and SAT/SBT failures can supplement assessment from clinical staff. This may decrease effort for clinical staff, especially when burden is high (e.g., ICU is near capacity). This may decrease the likelihood of poor patient outcomes in the event of workflow disruption (e.g., due to another patient experiencing an adverse event) while a SAT/SBT is being performed. An embodiment provides consistent assessment of pain and agitation, avoiding variability due to, for example, shift changes in clinical staff. Furthermore, continuous assessment of pain and agitation may capture intermittent signs that can be missed by clinical staff.

Illustrated in FIG. 3 is an example system for automated and continuous assessment of pain and agitation over an extended time period (e.g., several hours up to one day or more), with reporting of overall pain and agitation over this period (e.g., used to assess readiness for SATs/SBTs) and/or automated monitoring of pain and/or agitation over short time periods, with alerting of acute pain and/or agitation (used to monitor patients during SATs/SBTs, improving prompt detection of SAT/SBT failure or of various adverse events). In the example of FIG. 3, a camera 304 is capable of capturing imagery of a patient, e.g., full-body color video, and transmitting that imagery to other modules or devices, as necessary. Some embodiments may use a pan-tilt-zoom (PTZ) camera 304 capable of repositioning to capture full-body video of a patient. An embodiment may incorporate automatic positioning for control of the camera 304, for example using computer vision methods to detect the patient 301 and position or reposition the camera's 304 field of view so that the patient 301 is in the center of the video frame. Some embodiments may use a camera 304 that is part of another or a larger solution, for example integrated into an in-room device 305. In some embodiments, the camera 304 is a RGB (red, green, blue) camera. In some embodiments, the camera 304 is a RGB camera with near-infrared capability to enable operation under low-light conditions. In some embodiments, the camera 304 and associated video analysis are replaced or augmented by other non-contact sensors with associated signal analyses for detecting signs of pain and agitation. For example, supplemental or replacement non-contact sensors may include but are not limited to stereo cameras, depth sensing cameras, infrared cameras, light detection and ranging (LIDAR) devices, laser doppler velocimeters, mattress sensors, or combinations of the foregoing.

Vital signs are obtained from patient worn sensors 306 and respiration assessment data may be obtained via sensor(s) associated with endotracheal tube 303, e.g., an accelerometer or inertial management unit (IMU). Embodiments may incorporate measurements performed automatically by sensors incorporated into a ventilator, such as end-tidal CO₂. The vital signs assessed may vary in different embodiments. Embodiments may produce periodic, intermittent, or continuously updated summary assessments (e.g., continuous waveforms). The accuracy and performance of components such as the pain and agitation assessment may vary depending on vital signs available. Sensor data may be communicated to an appropriate system for evaluation either locally or remotely, or a combination of the foregoing.

An in-room device 305 may store relevant information, including sensor data and electronic health record (EHR) data and be configured to query a subset of medical chart data for a patient to form SAT/SBT safety screening assessments and SAT/SBT pass/failure assessments. Information queried from an EHR may include (not an exhaustive list) sedation and pain medication as well as the status of underlying conditions which (if unresolved) may cause a patient to be ineligible for SAT/SBTs. Some embodiments may retrieve information from an EHR via interoperability standards such as fast health interoperability resources (FHIR). As further described herein, some embodiments may be partly or completely implemented as components within the EHR.

As described herein, camera 304 may supply facial expression imagery to a device for detection of features or characteristics of interest via computer vision methods. Depending on the nature of the camera 304, this may be done locally (i.e., without sending imagery to a remote device or over a network such as the Internet) or this may be done remotely (i.e., calling an application programming interface of a machine learning model service for detection of facial features of interest) or a combination of local and remote processing may be used. In an embodiment where imagery is to be transmitted to a remote device, the imagery may be de-identified or anonymized prior to transmission or communication outside of a local system. For example, an embodiment may remove or mask certain parts of the imagery, e.g., parts of patient image containing personally identifiable information but not useful in assisting a model in making a classification. In any case, image or other sensor data may be encrypted or anonymized when at rest or in transit.

A system component performing pain or agitation assessment, e.g., camera 304, in-room device 305, a remote device, etc., which takes input from assessments of facial expression, body posture and movement, as well as from vital signs and chart data, provides output data to an endpoint such as a monitor, display or like component 302 capable of displaying information on pain and agitation over time. The output may include output to a device such as a clinician mobile device capable of generating an alert or displaying information on acute pain and/or agitation.

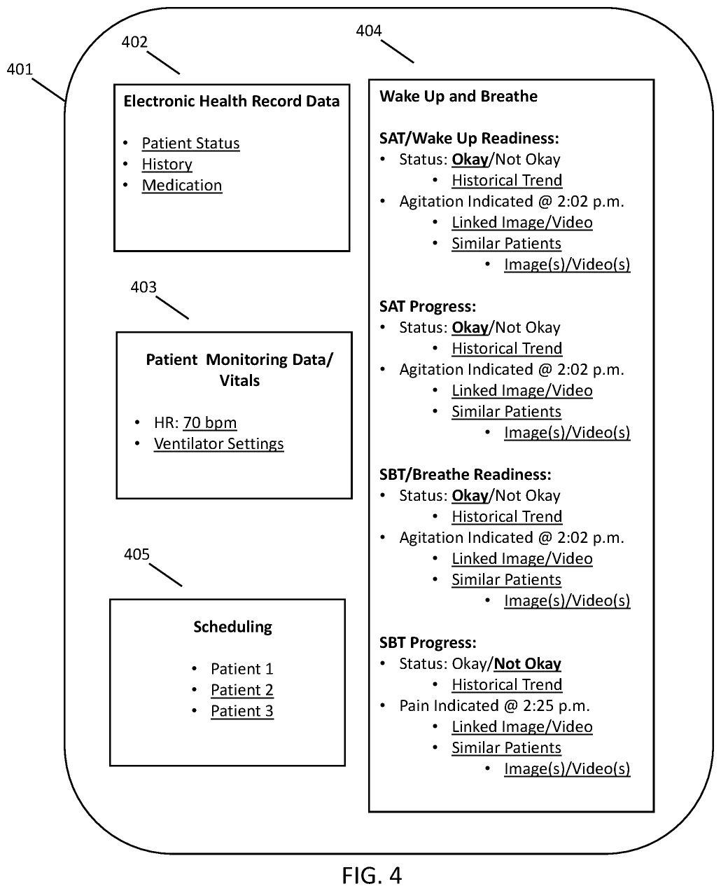

As shown in FIG. 4, an embodiment may provide a display interface 401, e.g., via monitor 302, that includes data relevant for a clinician conducting a wake up and breathe protocol, including indications such as indications 102a-d or data derived therefrom. In the example of FIG. 4, a display component 402 includes relevant data from an EHR, e.g., patient status, history of conditions and medications, etc. Some or all information in display component 402 may be retrieved automatically from an EHR or it may be retrieved in response to manual input, e.g., selections from a clinician. In an embodiment the patient status input may be part of an EHR interface, or it may be a separate interface. The display interface 401 of FIG. 4 may also include a display component 403 that includes patient monitoring data or vitals, e.g., as derived from patient worn sensors. This information of display component 403 may include vital information such as HR or data related to ventilation, e.g., FIO₂, PEEP, etc.

Still referring to FIG. 4, some embodiments may use expert-authored rules, e.g., to reproduce a protocol such as the “wake up and breathe” protocol shown above in FIG. 1. In this regard, display interface 401 may include logical elements in a display component 404 that facilitate adherence to the relevant protocol or pats thereof. In the example of FIG. 4, display component 404 includes wake up and breathe information, displayed in a logical fashion to facilitate a clinician's quick review and use of the same. As illustrated in the example of FIG. 4, display component 404 includes one or more of SAT/Wake Up Readiness, SAT Progress, SBT/Breathe Readiness, and SBT Progress data. In subcategories nested information is provided with an interactive display element for access. For example, a top level of information displays the overall judgment as to the protocol stage's state, e.g., okay for SAT readiness. A user may review and interact with the display to obtain more information, e.g., via linked informational elements, as displayed. In the example of FIG. 4, the display component 404 provides a link in the status display element, a link in a nested historical trend element, a link in the agitation element, a link in the first nested level elements for linked images and video and similar patients, as well as nested images and videos thereof. This provides the user with an interactive interface that can be used to quickly see the patient status according to inputs from clinicians and automated programs, such as facial image analysis as described herein, review historical data related to the patient and similar patients, as well as access the underlying data on which the determinations were made. For example, in FIG. 4 the display component 404 may be quickly reviewed to identify that the patient has successfully progressed through SAT safety screening, SAT, and SBT safety screening, but a failure indication is present for SBT progress due to an acute detection of pain, with the imagery accessible via an interactive display element.

Further, display interface 401 may include a display component 405 that provides patient scheduling information or access thereto. In the example illustrated, display component 405 has organized patients 1-3 in order of priority for attempting SAT, e.g., based on readiness scores provided using an embodiment. The clinician can quickly ascertain which patients are available for review and may proceed to remaining patients, e.g., patient 2, in a coordinated fashion after completion of a current patient, e.g., patient 1. Some embodiments may store the result of an assessment in an EHR.

In some embodiments, monitoring and alerting components may be implemented as part of an EHR interface and alerts or status information provided to various endpoints such as clinician devices. Some embodiments may provide an interface similar to display interface 401 with different information, for example less detailed protocol information and more detailed scheduling information (e.g., readiness scores) for a group of patients (e.g., one ward) in a single interface. This may include sorting patients according to a metric such as readiness score (e.g., to easily identify patients suitable for SATs/SBTs, to easily identify patients with high pain and/or agitation during a SAT/SBT and are suitable for clinician monitoring and confirmation, etc.) or other information visualization capabilities.

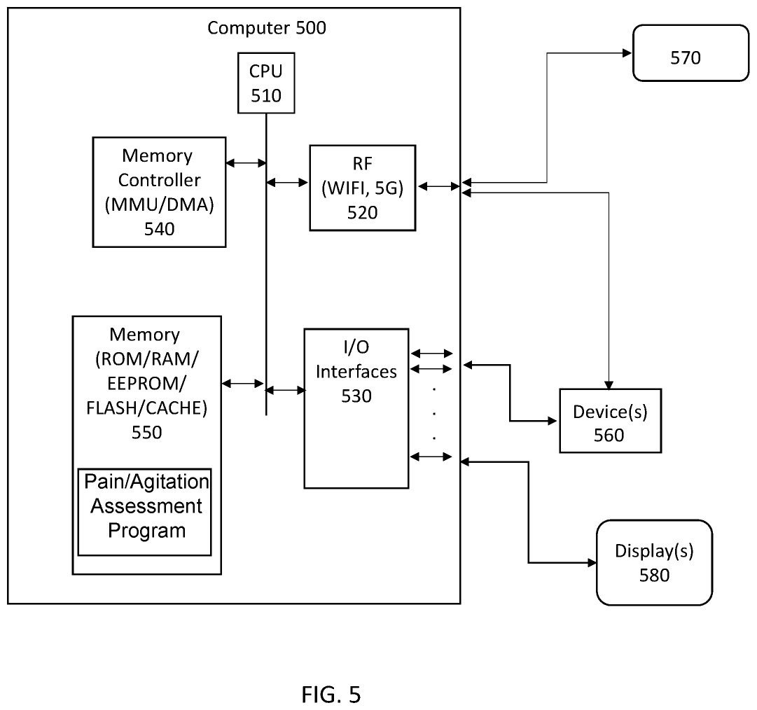

Referring to FIG. 5, it will be readily understood that certain embodiments can be implemented using any of a wide variety of devices or combinations of devices and components. In FIG. 5 an example of a computer 500 and its components are illustrated, which may be used in a device for implementing the functions or acts described herein, e.g., performing computer vision analysis of a patient as part of a wake up and breathe protocol. In addition, circuitry other than that illustrated in FIG. 5 may be utilized in one or more embodiments. The example of FIG. 5 includes certain functional blocks, as illustrated, which may be integrated onto a single semiconductor chip to meet specific application requirements.

One or more processing units are provided, which may include a central processing unit (CPU) 510, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 510 may comprise a single integrated circuit comprising several units, the design and arrangement of which vary according to the architecture chosen.

Computer 500 also includes a memory controller 540, e.g., comprising a direct memory access (DMA) controller to transfer data between memory 550 and hardware peripherals such as camera 570. Memory controller 540 includes a memory management unit (MMU) that functions to handle cache control, memory protection, and virtual memory. Computer 500 may include controllers for communication using various communication protocols (e.g., I²C, USB, etc.).

Memory 550 may include a variety of memory types, volatile and nonvolatile, e.g., read only memory (ROM), random access memory (RAM), electrically erasable programmable read only memory (EEPROM), Flash memory, and cache memory. Memory 550 may include embedded programs, code and downloaded software, e.g., artificial neural network program(s) trained using select patient images useful in producing predetermined classifications for use in SAT/STB protocols as described herein. By way of example, and not limitation, memory 550 may also include an operating system, application programs, other program modules, code and program data, which may be downloaded, updated, or modified via remote devices.

A system bus permits communication between various components of the computer 500. I/O interfaces 530 and radio frequency (RF) devices 570, e.g., WIFI and telecommunication radios, may be included to permit computer 500 to send and receive data to and from remote devices using wireless mechanisms, noting that data exchange interfaces for wired data exchange may be utilized. Computer 500 may operate in a networked or distributed environment using logical connections to one or more other remote computers or databases. The logical connections may include a network, such local area network (LAN) or a wide area network (WAN) but may also include other networks/buses. For example, computer 500 may communicate data with and between a device 560 running one or more artificial neural networks, training programs for training the same, and other devices, e.g., display(s) 580, e.g., an in-room system, ventilator, clinician mobile device, ward device, etc. that uses data such as classification outputs, readiness scores, scheduling data, etc., as described herein. It will be appreciated by those having skill in the art that artificial neural networks such as those described herein, once trained, may be provided, and used on a local device, e.g., computer 500, which may take the form of an end user device such as a smartphone, tablet, desktop computer, camera, in-room device, etc.

Computer 500 may therefore execute program instructions or code configured to obtain, store, and analyze patient imagery data and perform other functionality of the embodiments, as described herein. A user can interface with (for example, enter commands and information) the computer 500 through input devices, which may be connected to I/O interfaces 530. A display or other type of device may be connected to the computer 500 via an interface selected from I/O interfaces 530.

It should be noted that the various functions described herein may be implemented using instructions or code stored on a memory, e.g., memory 550, that are transmitted to and executed by a processor, e.g., CPU 510. Computer 500 includes one or more storage devices that persistently store programs and other data. A storage device, as used herein, is a non-transitory computer readable storage medium. Some examples of a non-transitory storage device or computer readable storage medium include, but are not limited to, storage integral to computer 500, such as memory 550, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method, comprising:
obtaining, using a set of one or more processors, a patient state indicating that a patient is associated with one or more of a spontaneous awakening trial (SAT) and a spontaneous breathing trial (SBT);
obtaining, using a set of one or more sensors, imagery of the patient associated with one or more of the SAT and the SBT;
evaluating, using the set of one or more processors, the imagery of the patient to classify the imagery as indicative of an observation item associated with one or more of the SAT and SBT; and
providing, using the set of one or more processors, an indication based on the evaluating.

2. The method of claim 1, wherein the imagery comprises facial imagery.

3. The method of claim 2, wherein the evaluating comprises classifying the facial imagery as indicative of an observation item including one or more of pain, agitation, and anxiety.

4. The method of claim 1, wherein the imagery is obtained in a continuous manner during a protocol time window.

5. The method of claim 1, wherein:
the obtaining, using the set of one or more sensors, comprises obtaining sensor data indicative of one or more of patient movement and body posture; and
the evaluating further comprises using the sensor data and the imagery to classify the patient for readiness for one or more of the SAT and the SBT.

6. The method of claim 1 wherein:
the obtaining, using the set of one or more sensors, comprises obtaining sensor data indicative of one or more of patient movement and body posture; and
the evaluating further comprises using the sensor data and the imagery to classify the patient as failing one or more of the SAT and the SBT.

7. The method of claim 1, further comprising displaying the indication or data derived therefrom in an interactive display, wherein the interactive display comprises a display component having one or more interactive elements associated with one or more of the SAT and the SBT.

8. The method of claim 7, wherein the one or more interactive elements comprise a link to the imagery or data derived therefrom.

9. The method of claim 7, wherein the displaying the indication comprises incorporating the indication into an electronic health record (EHR).

10. The method of claim 1, wherein the providing the indication comprises providing the indication to an end point including one or more of an in-room patient display, a clinician mobile device, a centralized monitoring station for an ICU, a remotely located telemedicine facility, and a scheduling system.

11. A device, comprising:
a set of one or more processors; and
a set of one or more memory devices operatively coupled to the set of one or more processors and storing code executable by the set of one or more processors to:
obtain a patient state indicating that a patient is associated with one or more of a spontaneous awakening trial (SAT) and a spontaneous breathing trial (SBT);
obtain imagery of the patient associated with one or more of the SAT and the SBT;
evaluate the imagery of the patient to classify the imagery as indicative of an observation item associated with one or more of the SAT and SBT; and
provide an indication based on evaluating the imagery.

12. The device of claim 11, wherein the imagery comprises facial imagery.

13. The device of claim 12, wherein to evaluate comprises classifying the facial imagery as indicative of an observation item including one or more of pain, agitation, and anxiety.

14. A computer program product, comprising:
a non-transitory storage medium comprising computer executable code, the computer executable code comprising:
code that obtains, using a set of one or more processors, a patient state indicating that a patient is associated with one or more of a spontaneous awakening trial (SAT) and a spontaneous breathing trial (SBT);
code that obtains imagery of the patient associated with one or more of the SAT and the SBT;
code that evaluates the imagery of the patient to classify the imagery as indicative of an observation item associated with one or more of the SAT and SBT; and
code that provides an indication based on evaluating the imagery.

15. The computer program product of claim 14, wherein the code that evaluates comprises code that classifies facial imagery as indicative of an observation item including one or more of pain, agitation, and anxiety.
